(19) United States
(12) Patent Application Publication
(10) Pub. No.: US 2024/0417758 A1
(43) Pub. Date: Dec. 19, 2024

(54) PROCESSES FOR FIXATION OF CARBON DIOXIDE

(71) Applicant: Jupeng Bio (HK) Limited, Hong Kong (CN)

(72) Inventors: Ryan Senaratne, Fayetteville, AR (US); Bruce Walker, Fayetteville, AR (US); Brandon Beard, Tontitown, AR (US); Melissa Chanderban, Lowell, AR (US)

(73) Assignee: Jupeng Bio (HK) Limited, Hong Kong (CN)

(21) Appl. No.: 18/740,693

(22) Filed: Jun. 12, 2024

Related U.S. Application Data
(60) Provisional application No. 63/521,323, filed on Jun. 15, 2023.

(51) Int. Cl. C12P 5/02; C12N 1/20; C12R 1/01

(52) U.S. Cl. CPC C12P 5/023; C12N 1/205; C12R 2001/01

(57) ABSTRACT

System and process are provided for fixation of carbon dioxide through fermentation. More specifically, the disclosure includes fermenting carbon dioxide into methane through methanogenic archaea. The disclosure further provides the integration of methanogenic fermentation with additional processes to achieve improved carbon efficiency.

Processes are provided for fixation of carbon dioxide. More specifically, the processes include fermenting carbon dioxide into methane and/or organic acids. The process may further include cracking methane to generate hydrogen or hydrogen and CO for subsequent fermentations or providing methane directly to a methylotroph fermentation process. Additional processes include processing cell mass from fermentations into single cell proteins to be used as nutrient supplements.

### BACKGROUND

Carbon monoxide and carbon dioxide emissions from industrial processes are two of the major drivers of climate change and global warming. Microbial fermentation can reduce such carbon emissions by utilizing microorganisms, through their metabolic pathways, to convert carbon monoxide (CO), hydrogen (H₂) and/or carbon dioxide (CO₂) into useful oxygenated hydrocarbon compounds, such as ethanol, butanol, acetate, butyrate, 2,3-butanediol, and other desired products.

Large scale microbial fermentation also produces large amount of microbial biomass. Traditionally, disposal of microbial biomass needs highly expensive waste treatment system, storage sites and landfills. Previously finding shows microbial biomass can be recovered into single cell protein (SCP) and other components for reuse as source of proteins, amino acids, and carbohydrates that are useful as a nutrient supplement for animals, plants, or human beings. For example, U.S. Pat. No. 10,856,560 describes a method of producing whole cell animal feed by culturing acetogens to produce microbial biomass.

Accordingly, there is a need for processes and systems which can effectively convert carbon dioxide into products for utilization in other processes. Further, there remains a need for a process and system for effectively converting microbial biomass into digestion friendly nutrient supplements, and compositions of any such nutrient supplements.

### SUMMARY

In one aspect, a process for converting CO₂ includes fermenting a gaseous substrate that includes CO₂ and H₂ with methanogenic archaea in a methanogen fermentation vessel; maintaining a CO₂ to H₂ ratio of about 1:3 to about 1:4 in the gaseous substrate; and recovering methane from the methanogen fermentation vessel.

In one aspect, a process for converting CO₂ includes fermenting a gaseous substrate that includes CO₂ and H₂ with methanogenic archaea in a methanogen fermentation vessel; recovering methane from the methanogen fermentation vessel; cracking at least a portion of the methane to generate H₂; and returning at least a portion of the H₂ to the methanogen fermentation vessel.

In another aspect, a process for converting CO and CO₂ includes fermenting a gaseous substrate that includes CO and H₂ with CO converting acetogenic bacteria in a CO fermentation vessel to produce an alcohol and a CO₂ containing vent gas; providing the CO₂ containing vent gas from the CO fermentation vessel to a methanogen fermentation vessel; and fermenting the CO₂ containing vent gas with methanogenic archaea in the methanogen fermentation vessel to produce methane.

In another aspect, a process for converting CO and CO₂, the process includes fermenting a gaseous substrate that includes CO and H₂ with CO converting acetogenic bacteria in a CO fermentation vessel to produce an alcohol and a first CO₂ containing vent gas and providing the CO₂ containing vent gas from the CO fermentation vessel to an acetogenic CO₂ fermentation vessel. The process further comprises fermenting the first CO₂ containing vent gas with CO₂ converting acetogenic bacteria in the acetogenic CO₂ fermentation vessel to produce an organic acid and a second CO₂ containing vent gas; providing the organic acid to the CO fermentation vessel and the second CO₂ containing vent gas to a methanogen fermentation vessel; and fermenting the second CO₂ containing vent gas with methanogenic archaea in the methanogen fermentation vessel to produce methane.

In another aspect, a process for converting CO and CO₂, the process includes fermenting a gaseous substrate that includes CO and H₂ with CO converting acetogenic bacteria in a CO fermentation vessel to produce an alcohol and a first CO₂ containing vent gas; providing at least a portion of the first CO₂ containing vent gas from the CO fermentation vessel to an acetogenic CO₂ fermentation vessel; fermenting the at least a portion of the first CO₂ containing vent gas with CO₂ converting acetogenic bacteria in the acetogenic CO₂ fermentation vessel to produce an organic acid; providing the organic acid to the CO fermentation vessel; providing at least another portion of the first CO₂ containing vent gas from the CO fermentation vessel to a methanogen fermentation vessel; and fermenting the at least another portion of the first CO₂ containing vent gas with methanogenic archaea in a methanogen fermentation vessel to produce methane.

### BRIEF DESCRIPTION OF FIGURES

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 illustrates a process for converting CO₂ that includes methane cracking to convert methane into hydrogen and solid carbon and/or methane reforming to produce hydrogen and CO.

FIG. 2 shows a process for converting CO and CO that includes fermentation with methanogenic archaea, and fermentation with CO converting acetogenic bacteria.

FIG. 3 shows a process for converting CO and CO₂ that includes fermentation with methanogenic archaea, fermentation with CO₂ converting acetogenic bacteria and fermentation with CO converting acetogenic bacteria.

### DETAILED DESCRIPTION

The following description is not to be taken in a limiting sense, but is made merely for the purpose of describing the general principles of exemplary embodiments. The scope of the disclosure should be determined with reference to the claims.

The term “about” modifying any amount refers to the variation in that amount encountered in real world conditions, e.g. in the lab, pilot plant, or production facility. For example, an amount of an ingredient or measurement employed in a mixture or quantity when modified by “about” includes the variation and degree of care typically employed in measuring in an experimental condition in production plant or lab. For example, the amount of a component of a product when modified by “about” includes the variation between batches in multiple experiments in the plant or lab and the variation inherent in the analytical method. Whether or not modified by “about” the amounts include equivalents to those amounts. Any quantity stated herein and modified by “about” can also be employed in the present disclosure as the amount not modified by “about”.

The use of the terms “a”, “an”, “the” and similar referents in the context of this disclosure are to be construed to cover both the singular and the plural, unless otherwise indicated or clearly contradicted by context.

Unless otherwise indicated, the terms “comprising”, “including”, “having”, “containing”, or “characterized by” are inclusive and does not exclude any additional, unrecited elements or method steps (i.e. meaning “including, but not limited to”). The use of any examples or exemplary language (e.g., “such as”, “for example”, “for instance”) provided herein is intended merely to illuminate the disclosure and does not impose a limitation on the scope of the disclosure unless otherwise claimed.

Fermentation is a metabolic process used by microorganisms to generate energy for cell growth. Certain microorganism can ferment a C1-containing gaseous substrate, such as syngas, carbon monoxide (CO) containing gaseous substrate, or carbon dioxide (CO₂) containing gaseous substrate, to sustain their growth and produce oxygenated hydrocarbon compounds. In such cases, the microorganism uses the one or more C1 components in the C1-containing gaseous substrate as the primary carbon source for its growth. The terms “fermentation”, “fermentation process”, “microbial fermentation process” and the like are intended to encompass both the growth phase and the product biosynthesis phase of the process. During an anaerobic microbial fermentation process, large amounts of microbial biomass are obtained, which may be purged out and processed into useful products, such as nutrient supplements. Specifically, the present disclosure includes a process of extracting nutrient supplements out of microbial biomass from an anaerobic fermentation process.

Fermentable gaseous substrate refers to C1-containing gaseous substrate comprises one or more of CO, CO₂, or CH₂O₂. Suitable gaseous substrate may include various synthesis gas (i.e. syngas) and industrial off-gas.

Syngas may be provided from any known source. In one aspect, syngas may be sourced from gasification of carbonaceous materials. Gasification involves partial combustion of biomass in a restricted supply of oxygen. The resultant gas may include CO, CO₂, and H₂. Some examples of suitable gasification methods and apparatus are provided in U.S. Ser. Nos. 61/516,667, 61/516,704 and 61/516,646, all of which were filed on Apr. 6, 2011, and in U.S. Ser. Nos. 13/427,144, 13/427,193 and 13/427,247, all of which were filed on Mar. 22, 2012, and all of which are incorporated herein by reference. In another aspect, syngas may be generated from electrolysis of water and carbon dioxide. In this aspect, oxygen is removed from the resultant gas and the resultant gas may be further blended with other gas sources to form a desired fermentable gaseous substrate.

Industrial off-gas may include the C1-containing waste gas from industrial processes that would otherwise be exhausted into the atmosphere. Examples of industrial off-gas include gases produced during microbial fermentation, ferrous metal products manufacturing, non-ferrous products manufacturing, petroleum refining processes, gasification of coal, electric power production, carbon black production, ammonia production, methanol production, coke manufacturing and gas reforming.

The C1-containing gaseous substrate may include H₂. He may also be separately supplemented into the C1-containing gaseous substrate to form desired gas composition suitable for fermentation. Examples of H₂ sources include gases produced during ferrous metal products manufacturing, non-ferrous products manufacturing, petroleum refining processes, gasification of coal, gasification of biomass, electric power production, carbon black production, ammonia production, methanol production and coke manufacturing. Other sources of hydrogen may include for example, H₂O electrolysis and bio-generated H₂.

The fermentation of the fermentable gaseous substrate with the microorganism takes place in a fermentation vessel. Fermentation vessel includes a fermentation bioreactor consisting of one or more vessels and/or towers or piping arrangements, which includes a batch reactor, semi-batch reactor, continuous reactor, continuous stirred tank reactor (CSTR), bubble column reactor, external circulation loop reactor, internal circulation loop reactor, immobilized cell reactor (ICR), trickle bed reactor (TBR), moving bed biofilm reactor (MBBR), gas lift reactor, membrane reactor such as hollow fiber membrane bioreactor (HFMBR), static mixer, gas lift fermentor, or other vessel or other device suitable for gas-liquid contact.

A culture medium suitable for anaerobic microbial growth and fermenting fermentable gaseous substrate into one or more oxygenated hydrocarbon compounds can be added to the fermentation vessel to support the fermentation of the gaseous substrate by the acetogenic bacteria. Some examples of medium compositions are described in U.S. Ser. Nos. 16/530,502 and 16/530,481, filed Aug. 2, 2019, and in U.S. Pat. No. 7,285,402, filed Jul. 23, 2001, all of which are incorporated herein by reference. The medium may be sterilized to remove undesirable microorganisms and the fermentation vessel is inoculated with the desired microorganisms. Sterilization may not always be required. Suitable culture medium for methanogen fermentation is described in U.S. Pat. No. 11,401,499 which is incorporated herein by reference.

### Methanogenic Fermentation and Methane Cracking

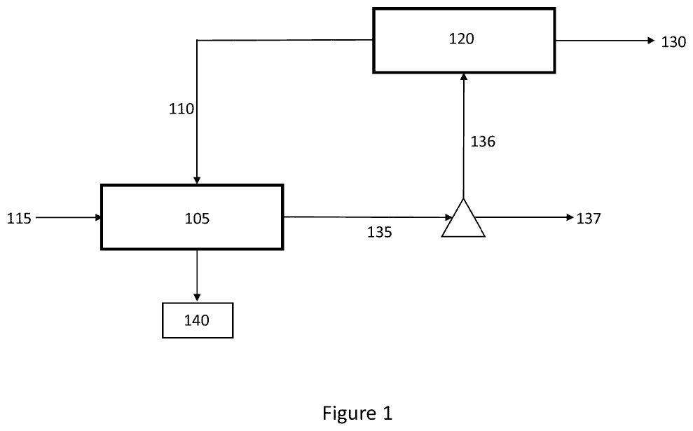

A process for converting CO₂ as illustrated in FIG. 1 includes the methane production in the methanogen fermentation vessel 105 and the use of methane cracker 120 to convert the methane 135 into hydrogen 110 and solid carbon or carbon monoxide 130.

Methane Production: In one aspect illustrated in FIG. 1, a process includes a methanogen fermentation vessel 105 that can be integrated with industrial processes that produce CO₂. In this aspect, the methanogen fermentation vessel 105 contains a microbial culture capable of hydrogenotrophic methanogenesis (i.e. the conversion of CO₂ plus H₂ to methane). The methanogen fermentation vessel 105 is coupled to a hydrogen source and a CO₂ gas source 115. The hydrogen source may be the hydrogen rich stream 110 produced by methane cracker 120. A separate hydrogen source may be provided to the methanogen fermentation vessel 105. H₂ and CO₂ may each be added separately to the methanogen fermentation vessel 105 or blended together and then added to the methanogen fermentation vessel 105. The process includes maintaining a ratio of CO₂ to H₂ in the fermentation vessel 105 of about 1:5 to about 1:1, in another aspect, about 1:5 to about 1:2, in another aspect, about 1:5 to about 1:3, in another aspect, about 1:5 to about 1:4, in another aspect, about 1:4 to about 1:1, in another aspect, about 1:4 to about 1:2, in another aspect, about 1:4 to about 1:3, in another aspect, about 1:3 to about 1:1, in another aspect, about 1:3 to about 1:2, and in another aspect, about 1:2 to about 1:1. Total gas delivery rates in the range of about 0.2 to about 25 volume of gas, in another aspect, about 2 to about 16, in another aspect, about 1 to 22, and in another aspect, about 0.5 to 20 (STP, standard temperature and pressure) per volume of culture per minute are suitable. The methanogen fermentation vessel 105 may also produce a fermentation liquid broth containing methanogen archaea 140 that may be processed into single cell protein.

Suitable microbial cultures are readily obtainable from public collections of microorganisms or can be isolated from a variety of environmental sources. Such environmental sources include anaerobic soils and sands, bogs, swamps, marshes, estuaries, dense algal mats, both terrestrial and marine mud and sediments, deep ocean and deep well sites, sewage and organic waste sites and treatment facilities, and animal intestinal tracts and feces. Many pure cultures of single species are suitable. Classified pure cultures are all members of the Archaeal domain [Woese et al. Proc Natl Acad Sci USA 87:4576-4579 (1990) “Towards a natural system of organisms: Proposal for the domains Archaea, Bacteria, and Eucharya.”, incorporated herein by reference] and fall within 4 different classes of the Euryarchaea kingdom. Examples of suitable organisms have been classified into 4 different genera within the Methanobacteria class (e.g. Methanobacterium alcaliphilum, Methanobacterium bryantii, Methanobacterium congolense, Methanobacterium defluvii, Methanobacterium espanolae, Methanobacterium formicicum, Methanobacterium ivanovii. Methanobacterium palustre, Methanobacterium thermaggregans, Methanobacterium uliginosum, Methanobrevibacter acididurans, Methanobrevibacter arboriphilicus, Methanobrevibacter gottschalkii, Methanobrevibacter olleyae, Methanobrevibacter ruminantium, Methanobrevibacter smithii, Methanobrevibacter woesei, Methanobrevibacter wolinii, Methanothermobacter marburgensis, Methanothermobacter thermautotrophicum (also known as Methanothermobacter thermoautotroiphicus), Methanothermobacter thermoflexus, Methanothermobacter thermophilus, Methanothermobacter wolfeii, Methanothermus sociabilis), 5 different genera within the Methanomicrobia class (e.g. Methanocorpusculum bavaricum, Methanocorpusculum parvum, Methanoculleus chikuoensis, Methanoculleus submarinus, Methanogenium frigidum, Methanogenium liminatans, Methanogenium marinum, Methanosarcina acetivorans, Methanosarcina barkeri, Methanosarcina mazei, Methanosarcina thermophila, Methanomicrobium mobile), 7 different genera within the Methanococci class (e.g. Methanocaldococcus jannaschii, Methanococcus aeolicus, Methanococcus maripaludis, Methanococcus vannielii, Methanococcus voltaci, Methanothermococcus thermolithotrophicus, Methanocaldococcus fervens, Methanocaldococcus indicus, Methanocaldococcus infernus, Methanocaldococcus vulcanius), and one genus within the Methanopyri class (e.g. Methanopyrus kandleri). Suitable cultures are available from public culture collections (e.g. the American Type Culture Collection, the Deutsche Sammlung von Mikroorganismen and Zellkulturen GmbH, and the Oregon Collection of Methanogens). Many suitable hydrogenotrophic methanogens, isolated in pure culture and available in public culture collections, have not yet been fully classified. Preferred pure culture organisms include Methanosarcinia barkeri. Methanococcus maripaludis, Methanothermobacter thermoautotrophicus, and Methanothermobacter marburgensis.

Suitable cultures of mixtures of two or more microbes are also readily isolated from the specified environmental sources [Bryant et al. Archiv Microbiol 59:20-31 (1967) “Methanobacillus omelianskii, a symbiotic association of two species of bacteria.”, incorporated herein by reference]. Suitable mixtures may be consortia in which cells of two or more species are physically associated or they may be syntrophic mixtures in which two or more species cooperate metabolically without physical association. Mixed cultures may have useful properties beyond those available from pure cultures of known hydrogenotrophic methanogens. These properties may include, for instance, resistance to contaminants in the gas feed stream, such as oxygen, ethanol or other trace components, or aggregated growth, which may increase the culture density and volumetric gas processing capacity of the culture.

Suitable cultures of mixed organisms may also be obtained by combining cultures isolated from two or more sources. One or more of the species in a suitable mixed culture should be an Archaeal methanogen. Any non-Archaeal species may be bacterial or eukaryotic.

Suitable cultures may also be obtained by genetic modification of non-methanogenic organisms in which genes essential for supporting hydrogenotrophic methanogenesis are transferred from a methanogenic microbe or from a combination of microbes that may or may not be methanogenic on their own. Suitable genetic modification may also be obtained by enzymatic or chemical synthesis of the necessary genes.

The methanogen fermentation vessel 105 provides continuous methane production using a continuous hydrogenotrophic methanogenic culture operating under stable conditions. An example of such suitable conditions is provided in Schill, N., van Gulik, M., Voisard, D., & von Stockar, U. (1996) Biotechnol & Biocng 51:645-658. “Continuous cultures limited by a gaseous substrate: development of a simple, unstructured mathematical model and experimental verification with Methanobacterium thermoautotrophicum”, incorporated herein by reference. Culture media may be comprised of dilute mineral salts, and should be adapted to the particular culture in use.

Concentrations of various medium components for use in the methanogen fermentation process are as follows:

| | Concentration | Feed Rate |
|---|---|---|
| Element | mg/L | μg/gram cells/min |
| NH₄⁺ | 82-3280 | 170-1,375 |
| Fe | 0.85-34 | 0.91-8.22 |
| Ni | 0.07-2.81 | 0.12-1.05 |
| Co | 0.037-1.49 | 0.13-0.66 |
| Zn | 0.45-23.8 | 0.08-0.56 |
| Mo | 0.003-0.397 | 0.012-0.13 |
| chelator | 2.5-100 | 2.65-17.37 |
| W | 0.8-32.1 | 0.94-8.06 |
| K | 98-3933 | 217-1,542 |
| Mg | 0.71-28.69 | 0.69-6.24 |
| Na | 875-35000 | 290-8,750 |
| S | 15-625 | 1.12-13.41 |
| P | 20-805 | 9.71-87.39 |

Medium in the methanogen fermentation vessel 105 should be replenished at a rate suitable to maintain a useful concentration of essential minerals and to eliminate any metabolic products that may inhibit methanogenesis. Dilution rates below 0.2 culture volume per hour are suitable, since they yield high volumetric concentrations of active methane generation capacity.

In one aspect, a redox potential is maintained below −400 mV or lower during methanogenesis. In another aspect, the redox potential is maintained below −300 mV or lower, in another aspect, below-200 mV, and in still another aspect, below −100 mV.

In another aspect, temperature of the culture is maintained near the optimum for growth of the organism used in the culture (e.g. about 35° C. to about 37° C. for mesophilic organisms such as Methanosarcinia barkeri and Methanococcus maripaludis or about 60°-65° C. for thermophiles such as Methanothermobacter thermoautotrophicus, and about 85° C. . . . 90° C. for organisms such as Methanocaldococcus jannaschii, Methanocaldococcus fervens, Methanocaldococcus indicus, Methanocaldococcus infernus, and Methanocaldococcus vulcanius). However, it is envisioned that temperatures above or below the temperatures for optimal growth may be used.

In another aspect, a reducing agent may be introduced into the fermentation process along with CO₂ and hydrogen. This reducing agent can suitably be hydrogen sulfide or sodium sulfide. Hydrogen itself can be used as a reductant to maintain the redox potential of the culture in the range (<−100 mV) necessary for optimum performance of hydrogenotrophic methanogenesis. Generally, hydrogen is provided in concentrations effective in allowing for at least a portion of the carbon dioxide in the bioreactor to be converted into methane. In another aspect, the redox potential of the culture can be maintained at <−100 mV via an electrochemical cell immersed in the medium.

In another aspect, the process includes various methods and/or features that reduce the presence of oxygen in the CO₂ stream that is fed into the bioreactor. When obligate anaerobic methanogenic archaea are used to catalyze methane formation, the presence of oxygen may be detrimental to the performance of the process and contaminates the product gas. Therefore, the reduction of the presence of oxygen in the CO₂ stream is helpful for improving the process. In one aspect, the oxygen level is reduced prior to entry of the gas into the fermentation vessel by passing the mixed H₂/CO₂ stream over a palladium catalyst, which converts any trace oxygen to water. In this aspect, H₂ is provided in an amount above the amount needed in the culture by a 2:1 ratio relative to the contaminating oxygen. In another aspect, the oxygen is removed by pre-treatment of the gas stream in a bioreactor. In this aspect, the reductant may be provided either by provision of a source of organic material (e.g. glucose, starch, cellulose, fermentation residue from an ethanol plant, whey residue, etc.) that can serve as substrate for an oxidative fermentation. The microbial biological catalyst is chosen to oxidatively ferment the chosen organic source, yielding CO₂ from the contaminant oxygen. In this embodiment, additional H₂ would be provided to enable conversion in the anaerobic fermentor of this additional CO₂ to methane.

The methanogen fermentation process provides a specific CO₂ uptake of about 0.5 to about 3 mmol CO₂/minute/gram of cells, in another aspect, about 1 to about 2 mmol CO₂/minute/gram of cells, in another aspect, about 0.5 to about 1 mmol CO₂/minute/gram of cells, in another aspect, about 1 to about 3 mmol CO₂/minute/gram of cells, and in another aspect, about 0.5 to about 2 mmol CO₂/minute/gram of cells. In this aspect, the methanogen fermentation process is effective for providing a CO₂ conversion rate of 65% or more, in another aspect, 70% or more, in another aspect, 75% or more, in another aspect, 80% or more, in another aspect, 85% or more, in another aspect, 90% or more, in another aspect, 85% to 95%, and in another aspect, 90% to 99%. The process further provides a cell density of up to 100 g/L, in one aspect, 10 to 80 g/L, in one aspect, 15 to 60 g/L, in one aspect, 20 to 50 g/L, in one aspect 10 to 30 g/L, in another aspect, 15 to 45 g/L.

The process further provides a specific H₂ uptake of about 3 to about 12 mmol H₂/minute/gram of cells, in another aspect, about 3 to about 10 mmol H₂/minute/gram of cells, in another aspect, about 3 to about 8 mmol H₂/minute/gram of cells, in another aspect, about 3 to about 6 mmol H₂/minute/gram of cells, in another aspect, about 4 to about 12 mmol H₂/minute/gram of cells, in another aspect, about 4 to about 10 mmol H₂/minute/gram of cells, in another aspect, about 4 to about 8 mmol H₂/minute/gram of cells, in another aspect, about 5 to about 12 mmol Ha/minute/gram of cells, in another aspect, about 5 to about 10 mmol H₂/minute/gram of cells, and in another aspect, about 5 to about 8 mmol H₂/minute/gram of cells.

The process further provides a cell retention time of about 5 to about 50 hours, in another aspect, about 5 to about 40 hours, in another aspect, about 5 to about 30 hours, in another aspect, about 5 to about 25 hours, in another aspect, about 5 to about 20 hours, in another aspect, about 5 to about 10 hours, in another aspect, about 5 to about 8 hours, and in another aspect, about 8 to about 15 hours.

The process further provides a methane productivity of about 0.4 to about 3 mmol methane/minute/gram of cells, in another aspect, about 0.4 to about 2 mmol methane/minute/gram of cells, in another aspect, in another aspect, about 0.4 to about 1 mmol methane/minute/gram of cells, in another aspect, about 1.0 to about 3 mmol methane/minute/gram of cells, in another aspect, about 1.0 to about 2.5 mmol methane/minute/gram of cells, in another aspect, about 1.0 to about 2.5 mmol methane/minute/gram of cells, and in another aspect, about 1.5 to about 2.5 mmol methane/minute/gram of cells. In this aspect, the process is effective for providing a methane effluent gas with more than 60% methane concentration, in another aspect, more than 65% methane concentration, in another aspect, more than 70% methane concentration, in another aspect, more than 75%, in another aspect, more than 80%, and in still another aspect, more than 85%.

Methane Cracking: As further illustrated in FIG. 1, methane 135 produced by fermentation vessel 105 may be conveyed via line 136 to a methane cracker 120 or conveyed to power various processes, and/or can be stored and sold as fuel (all referred to as 137). The methane cracker 120 may pyrolyze methane into hydrogen 110 and solid carbon 130 or reform methane into hydrogen 110 and carbon monoxide 130. As used herein, methane cracking may include both pyrolysis and reforming. Methane pyrolysis may be accomplished by known methane pyrolysis processes, including for example, microwave pyrolysis, molten metal pyrolysis, plasma arc pyrolysis, and combinations thereof. Examples of methane reforming include steam reforming, dry reforming and partial oxidation.

In one aspect, methane pyrolysis is conducted using microwave pyrolysis. Microwave pyrolysis is described in WO 2022/232942, published Nov. 10, 2022, which is incorporated herein by reference. The process includes providing a methane containing feedstock to a microwave inert reaction vessel or providing a methane containing feedstock to a reaction vessel that is both microwave-inert and radio wave inert. Inside the vessel there is solid carbon, an absence or negligible amount at most, of molecular oxygen. Water and molecular oxygen are not added to the vessel. The only source of water and molecular oxygen inside the reaction vessel should be the small amounts of water, molecular oxygen and oxygen-containing species (CO₂) found in the feedstock. The solid carbon is then exposed to microwaves, radio waves, or both microwaves and radio waves so that the carbon is at a temperature of at least 200 Kelvin. The hot carbon heats the gaseous hydrocarbons, thereby forming hydrogen and additional solid carbon. Hydrogen and solid carbon are separated and the hydrogen 110 may be provided back to the methanogen fermentation vessel 105.

In another aspect, methane pyrolysis is conducted using molten metal pyrolysis. In another aspect, methane pyrolysis is conducted using plasma arc pyrolysis.

### CO Fermentation and Methanogenic Fermentation.

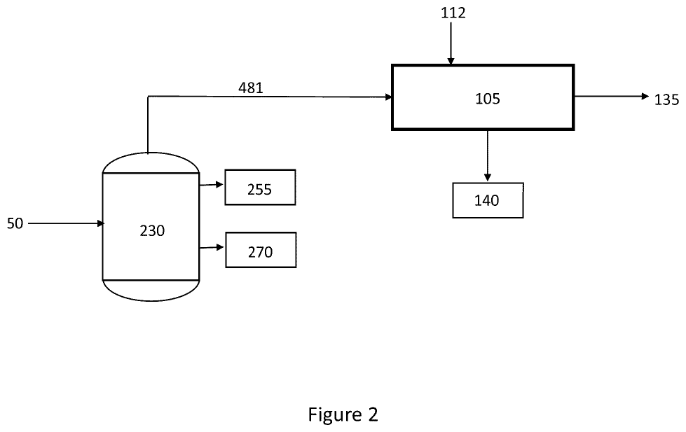

A process for converting CO and CO₂ as illustrated in FIG. 2 includes providing a CO containing gaseous substrate 50 to a CO fermentation vessel 230. CO converting acetogenic bacteria in the CO fermentation vessel 230 may convert CO into one or more alcohols 255. Vent gas 481 from the CO fermentation vessel 230 contains CO₂. The vent gas 481 may be provided to a methanogen fermentation vessel 105. A gaseous substrate 112 containing H₂ and/or additional CO₂ may also be provided to the methanogen fermentation vessel 105. Methanogenic archaea in the methanogen fermentation vessel convert CO₂ into methane 135. The methane 135 may be sold as “green methane/renewable natural gas” or may optionally be sent to methane cracking. In another aspect, H₂ and/or CO from methane cracking may be supplied to the methanogen fermentation vessel and/or CO fermentation vessel. Clostridium fermentation liquid broth 270 and/or methanogen fermentation liquid broth 140 may be processed into single cell protein.

Methane Production: In one aspect illustrated in FIG. 2, a process includes a methanogen fermentation vessel 105 as described herein with the description of FIG. 1.

Methane Cracking: In an optional aspect not shown in FIG. 2, a portion of methane 135 produced in the methanogen fermentation vessel 105 may be provided to a methane cracker as described herein in connection with FIG. 1. The methane cracker may be operated to provide a gaseous substrate that includes H₂ and/or CO. In one aspect, the H₂ and CO containing gaseous substrate may be separated into a H₂ rich stream and a CO rich syngas. The H₂ rich stream may be provided to the methanogen fermentation vessel 105 and the CO rich syngas may be provided to the CO fermentation vessel 230. In another aspect, the H₂ and CO containing gaseous substrate may be directly sent to the CO fermentation vessel 230. In still another aspect, no CO is produced from the methane cracker. In this scenario, the H₂ containing gaseous substrate produced from the methane cracker is directly sent to the methanogen fermentation vessel 105.

CO Fermentation: Certain acetogenic bacteria can ferment CO-containing gaseous substrate 50 in a CO fermentation vessel 230 into useful oxygenated hydrocarbon compounds 255, such as ethanol and butanol, and produce a fermentation liquid broth containing the acetogenic bacteria 270.

In this aspect, suitable gaseous substrate 50 contains at least about 5 mole % CO, in one aspect, at least about 10 mole %, in one aspect, at least about 20 mole %, in one aspect, at least about 30 mole %, in one aspect, about 10 to about 100 mole %, in another aspect, about 20 to about 100 mole % CO, in another aspect, about 30 to about 90 mole % CO, in another aspect, about 40 to about 80 mole % CO, and in another aspect, about 50 to about 70 mole % CO. In this aspect, the CO-containing gaseous substrate 50 may have about 40 mole % or less CO₂, in one aspect, the CO-containing gaseous substrate 50 may have about 30 mole % or less CO₂, in one aspect, the CO-containing gaseous substrate 50 may have about 20 mole % or less CO₂, in another aspect, the CO-containing gaseous substrate 50 may have about 10 mole % or less CO₂, in another aspect, the CO-containing gaseous substrate 50 may have about 1 mole % or less CO₂, in still another aspect, the CO-containing gaseous substrate 50 may comprise no or substantially no CO₂.

Depending on the composition of the CO-containing gaseous substrate 50, the CO-containing gaseous substrate 50 may be directly provided to the fermentation vessel 230 or may be further modified or blended to include an appropriate H₂ to CO molar ratio. In one aspect, the CO-containing gaseous substrate provided to the fermentation vessel has an H₂ to CO molar ratio of about 0.1 or more, in another aspect, about 0.2 or more, in another aspect, about 0.25 or more, and in another aspect, about 0.5 or more. In one aspect, H₂ and/or CO from a methane cracker may be supplied to the CO fermentation vessel 230.

Concentrations of various medium components for use in the CO bioconversion fermentation process are as follows:

| | Concentration | Feed Rate |
|---|---|---|
| Element | mg/L | μg/gram cells/min |
| NH₄⁺ | 164-6560 | 41-1640 |
| Fe | 1.7-68 | 0.425-17 |
| Ni | 0.07-2.81 | 0.017-0.702 |
| Co | 0.037-1.49 | 0.009-0.373 |
| Se | 0.027-1.1 | 0.006-0.274 |
| Zn | 0.116-4.64 | 0.198-5.95 |
| W | 0.8-32.1 | 0.26-8.03 |
| K | 39-1573 | 9.83-393.25 |
| Mg | 1.4-57.3 | 0.35-14.32 |
| S | 15-625 | 3.9-156.2 |
| P | 15-601 | 3.76-150.43 |
| d-biotin | 0.016-0.64 | 0.004-0.16 |
| thiamine HCl | 0.04-1.6 | 0.01-0.4 |
| calcium-D-pantothenate | 0.02-0.81 | 0.005-0.202 |

Examples of useful acetogenic bacteria for CO bioconversion fermentation process include Blautia producta, Butyribacterium methylotrophicum, Caldanaerobacter subterraneous, Caldanaerobacter subterraneous pacificus, Carboxydothermus hydrogenoformans, Clostridium aceticum, Clostridium acetobutylicum, Clostridium acetobutylicum P262, Clostridium autoethanogenum (DSM 19630 of DSMZ Germany), Clostridium autoethanogenum (DSM 10061 of DSMZ Germany), Clostridium autoethanogenum (DSM 23693 of DSMZ Germany), Clostridium autoethanogenum (DSM 24138 of DSMZ Germany), Clostridium carboxidivorans, Clostridium coskatii (ATCC PTA-10522), Clostridium drakei, Clostridium ljungdahlii PETC (ATCC 49587), Clostridium ljungdahlii ERI2 (ATCC 55380), Clostridium ljungdahlii C-01 (ATCC 55988), Clostridium ljungdahlii O-52 (ATCC 55889), Clostridium magnum, Clostridium pasteurianum (DSM 525 of DSMZ Germany), Clostridium ragsdalei P11 (ATCC BAA-622), Clostridium scatologenes, Clostridium thermoaceticum, Clostridium ultunense, Desulfotomaculum kuznetsovii, Eubacterium limosum, Geobacter sulfurreducens, Methanosarcina acetivorans, Methanosarcina barkeri, Oxobacter pfennigii, Peptostreptococcus productus, Clostridium Stick-landii, and mixtures thereof.

Anaerobic bacteria are bacteria that do not require oxygen for growth. An anaerobic bacteria may react negatively or even die if oxygen is present above certain threshold. Acetogenic bacteria are microorganisms that are capable of producing acetate under anaerobic respiration or fermentation by utilizing the Wood-Ljungdahl pathway as their main mechanism for energy conservation. Other useful oxygenated hydrocarbon compounds, such as formic acid, propionic acid, butyric acid, heptanoic acid, decanoic acid, ethanol, butanol, 2-butanol, and 2,3-butanediol, may also be produced by the acetogenic bacteria. Examples of the acetogenic bacteria suitable for converting C1-containing gaseous substrate to useful oxygenated hydrocarbon compounds include those of the genus Clostridium, such as strains of Clostridium ljungdahlii, including those described in WO 2000/68407, EP 117309, U.S. Pat. Nos. 5,173,429, 5,593,886 and 6,368,819, WO 1998/00558 and WO 2002/08438, strains of Clostridium autoethanogenum (DSM 10061 and DSM 19630 of DSMZ, Germany) including those described in WO 2007/117157 and WO 2009/151342 and Clostridium ragsdalei (P11, ATCC BAA-622) and Alkalibaculum bacchi (CP11, ATCC BAA-1772) including those described respectively in U.S. Pat. No. 7,704,723 and “Biofuels and Bioproducts from Biomass-Generated Synthesis Gas”, Hasan Atiyeh, presented in Oklahoma EPSCOR Annual State Conference, Apr. 29, 2010 and Clostridium carboxidivorans (ATCC PTA-7827) described in U.S. Patent Application No. 2007/0276447. Other suitable microorganisms includes those of the genus Moorella, including Moorella sp. HUC22-1, and those of the genus Carboxydothermus. Each of these references is incorporated herein by reference.

The CO fermentation may desirably be carried out under appropriate reaction conditions for the desired fermentation mode. For example, in one aspect, the CO fermentation can be set at a mode that focuses on CO-to-oxygenated hydrocarbon compounds (e.g. ethanol) production. In this mode, about 4% to 6% of the carbon from the CO fed to the CO fermentation is converted to biomass. In another aspect, the CO fermentation may be set at a mode that focuses on CO-to-microbial biomass production. In this mode, about 6% to 7.5% of the carbon from the CO fed to the CO fermentation is converted to biomass. Reaction conditions that should be considered include pressure, temperature, gas flow rate, liquid flow rate, medium pH, medium redox potential, agitation rate (if using a stirred tank reactor), inoculum level, appropriate gas substrate concentrations to ensure that CO in the liquid phase does not become limiting nor inhibitory, and appropriate product concentrations to avoid product inhibition. The CO fermentation process further provides a CO conversion rate of 80% or more, in one aspect, 85% or more, in one aspect, 90% or more, in another aspect, 80% to 99%, in another aspect, 85% to 98%, and in still another aspect 90% to 97%. A cell density of 5 g/L or more is maintained during the CO fermentation process, in one aspect, 10 g/L or more maintained, in one aspect, 12 g/L or more is maintained, in another aspect, 8 to 15 g/L is maintained, in another aspect, 10 to 20 g/L is maintained, in another aspect, 12 to 25 g/L is maintained, and in still another aspect, 10 to 30 g/L is maintained.

The CO fermentation further provides a specific alcohol productivity of about 10 grams alcohol/day/grams of cells or more, in another aspect, a specific alcohol productivity rate of about 12 g/day/grams of cells or more, in another aspect, a specific alcohol productivity rate of about 14 g/day/grams of cells or more, in another aspect, a specific alcohol productivity rate of about 10 to about 16 g/day/grams of cells, in another aspect, about 10 to about 14 g/day/grams of cells, in another aspect, about 10 to about 12 g/day/grams of cells, in another aspect, about 10 to about 16 g/day/grams of cells, in another aspect, about 10 to about 14 g/day/grams of cells, in another aspect, about 12 to about 16 g/day/grams of cells, and in another aspect, about 12 to about 14 g/day/grams of cells.

The CO converting acetogenic bacteria converts CO and produces one or more alcohols and a CO₂ containing vent gas. The CO₂ containing vent gas is then sent to the methanogenic fermentation vessel. The CO₂ containing vent gas contains 5% or less CO, in one aspect, 3% or less, in one aspect, 2% or less, and in another aspect, 1% or less. CO may be removed from the CO₂ containing vent gas before it enters the methanogenic fermentation vessel to avoid CO inhibition.

### CO Fermentation, Acetogenic CO₂ Fermentation and Methanogenic Fermentation

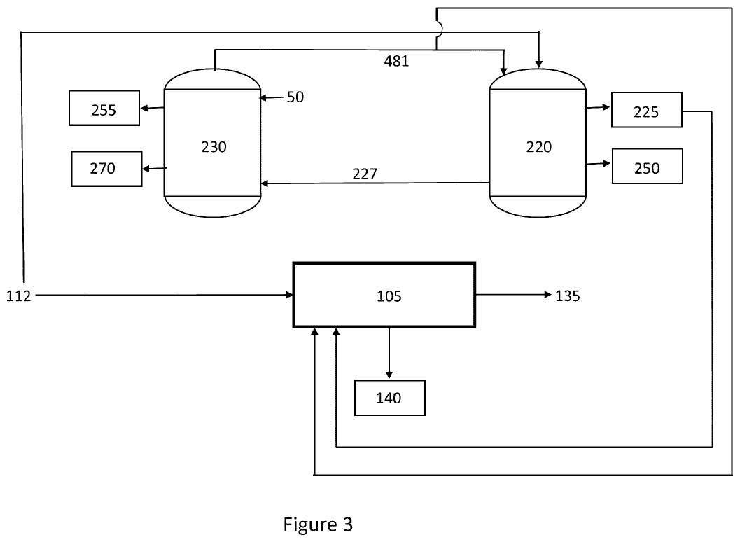

A process for converting CO and CO₂ as illustrated in FIG. 3 includes fermenting a gaseous substrate 50 that includes CO in a CO fermentation vessel 230. CO converting acetogenic bacteria in the CO fermentation vessel 230 converts CO into one or more alcohols 255. Vent gas 481 from the CO fermentation vessel 230 contains CO₂. The vent gas 481 may be provided to an acetogenic CO₂ fermentation vessel 220 and/or to a methanogenic fermentation vessel 105. The acetogenic CO₂ fermentation vessel 220 produces organic acid 227 which can be supplied to CO fermentation vessel 230 to increase the oxygenated hydrocarbon compounds 255, such as ethanol and butanol, productivity in the CO fermentation. Vent gas 225 from the acetogenic CO₂ fermentation vessel 220 may contain unconverted CO₂ and/or H₂, which may then be delivered to the methanogenic fermentation vessel 105. CO fermentation broth 270, acetogenic CO₂ fermentation broth 250, and/or methanogen fermentation broth 140 can be further purged out from the fermentation vessels and processed into single cell protein nutrient supplement.

Methane Production: In one aspect illustrated in FIG. 3 a process includes a methanogen fermentation vessel 105 as described herein with the description of FIG. 1. In this aspect, the methanogen fermentation vessel 105 may be used to balance organic acid consumption of the CO fermentation vessel 230 and CO₂ consumption of the in the acetogenic CO₂ fermentation vessel 220. A gaseous substrate 112 containing supplemented H₂ and/or external CO₂ may also be provided to the methanogen fermentation vessel 105 and/or the acetogenic CO₂ fermentation vessel 220.

CO-converting acetogenic bacteria and acetogenic CO, fermentation: CO₂ converting acetogenic bacteria may ferment CO₂-containing gaseous substrate into useful oxygenated hydrocarbon compounds, such as C1 to C10 organic acids 227, examples of which include acetic acid and butyric acid. In this aspect, the acetogenic CO₂ fermentation vessel may be supplied with supplemented H₂ 112. In one aspect, the organic acid 227 may be supplied to the CO fermentation vessel 230 and the vent gas 225 may be delivered to methanogen fermentation vessel 105. In one aspect, suitable CO₂-containing gaseous substrate contains at least about 10 mole % CO₂, in one aspect, at least about 20 mole %, in one aspect, at least about 30 mole %, in one aspect, at least about 40 mole %, in one aspect, about 10 to about 70 mole %, in another aspect, about 20 to about 70 mole % CO₂, in another aspect, about 30 to about 70 mole % CO₂, in another aspect, about 40 to about 70 mole % CO₂, in another aspect, about 10 to about 50 mole % CO₂, in another aspect, about 20 to about 40 mole % CO₂, and in still another aspect, about 30 to 50 mole % CO₂. In this aspect, the CO₂-containing gaseous substrate contains about 50 mole % or less CO, in one aspect, the CO₂-containing gaseous substrate contains about 40 mole % or less CO, in one aspect, the CO₂-containing gaseous substrate contains about 30 mole % or less CO, in one aspect, the CO₂-containing gaseous substrate contains about 20 mole % or less CO, in one aspect, the CO₂-containing gaseous substrate contains about 10 mole % or less CO, in one aspect, the CO-containing gaseous substrate contains about 5 mole % or less CO, in one aspect, the CO₂-containing gaseous substrate contains about 1 mole % or less CO, in another aspect, the CO₂-containing gaseous substrate contains no or substantially no CO.

Depending on the composition of the CO₂-containing gaseous substrate, the CO₂-containing gaseous substrate may be directly provided to an acetogenic CO₂ fermentation process or may be further modified or blended to include an appropriate H₂ to CO₂ molar ratio. For example, a stream comprising a high concentration of CO₂, such as the exhaust from an industrial process, can be combined with a stream comprising high concentrations of H₂, such as the off gas from a coke oven. In one aspect, the gaseous substrate provided to the fermentation vessel has an H₂ to CO₂ molar ratio of about 4:1 to about 1:2, in another aspect, about 4:1 to about 1:1, in another aspect, about 4:1 to about 2:1, and in still another aspect, about 3.5:1 to about 1.5:1.

Concentrations of various medium components for use in the CO₂ bioconversion fermentation process are as follows:

| | Concentration | Feed Rate |
|---|---|---|
| Element | mg/L | μg/gram cells/min |
| NH₄⁺ | 82-3280 | 20.5-820 |
| Fe | 0.85-34 | 0.28-8.5 |
| Ni | 0.07-2.81 | 0.023-0.702 |
| Co | 0.037-1.49 | 0.012-0.373 |
| Se | 0.027-1.1 | 0.009-0.274 |
| Zn | 0.59-23.8 | 0.198-5.95 |
| Mo | 0.003-0.397 | 0.003-0.1 |
| chelator | 2.5-100 | 0.83-25 |
| W | 0.8-32.1 | 0.26-8.03 |
| K | 98-3933 | 32.77-983.35 |
| Mg | 0.71-28.69 | 0.23-7.18 |
| Na | 875-35000 | 290-8750 |
| S | 15-625 | 2.08-62.5 |
| P | 20-805 | 6.7-201.3 |
| d-biotin | 0.016-0.64 | 0.005-0.16 |
| thiamine HCl | 0.04-1.6 | 0.01-0.4 |
| calcium-D-pantothenate | 0.02-0.81 | 0.006-0.202 |

Suitable acetogenic bacteria for CO₂ bioconversion includes a sodium pump which may also be described as sodium-translocating ATPases (for membrane bioenergetics). Sodium translocating ATPase are described in Müller, “Energy Conservation in Acetogenic Bacteria,” Appl. Environ. Microbiol. November 2003, vol. 69, no. 11, pp. 6345-6353, which is incorporated herein by reference. Acetogenic bacteria that include a sodium-translocating ATPase require about 500 ppm NaCl in their growth medium for growth. To determine if an acetogenic bacteria includes a sodium-translocating ATPase, the acetogen is inoculated into serum bottles containing about 30 to about 50 ml of growth medium with about 0 to about 2000 ppm NaCl. Normal growth at NaCl concentrations of about 500 ppm or more means that the acetogenic bacteria includes a sodium-translocating ATPase. In this aspect, the composition of the fermentation medium also includes a sodium ion concentration of about 40 to about 500 mmol per liter, in another aspect, about 40 to about 250 mmol per liter and in another aspect, a sodium ion concentration of about 50 to about 200 mmol per liter. In one aspect, the sodium ion concentration is about 500 ppm to about 8000 ppm, in another aspect, about 1000 ppm to about 7000 ppm, in another aspect, about 3000 ppm to about 6000 ppm, in another aspect, about 2000 to about 5000 ppm, and in another aspect, about 3000 to about 4000 ppm.

Examples of useful CO₂ converting acetogenic bacteria for CO₂ bioconversion include, Acetogenium kivui, Acetoanaerobium noterae, Acetobacterium woodii, Alkalibaculum bacchi CP11 (ATCC BAA-1772), Moorella thermoacetica, Moorella thermoautotrophica, Ruminococcus productus, Acetogenium kivui, and combinations thereof.

Acetogenic bacteria for CO₂ fermentation are able to produce C1 to C10 organic acids. In one aspect, the organic acid is acetic acid. In another aspect, the organic acid is butyric acid. In still another aspect, the organic acid is acetic acid or butyric acid, or a mixture of both. The fermentation process provides a simultaneous approach of generating a high specific productivity of oxygenated hydrocarbonaceous compound production while producing nutrient supplement from the bacterial cells used in the fermentation process. As used herein, specific productivity is expressed as specific STY. In this aspect, specific oxygenated hydrocarbon compound productivity may be expressed as specific STY (e.g. specific space time yield can be expressed as g alcohol/day/gram of cells or g organic acid/day/gram of cells). In one aspect, the fermentation process provides a specific organic acid productivity of about 0.2 to about 50 grams organic acid/day/gram of cells, in another aspect, about 0.2 to about 20 grams organic acid/day/gram of cells, in another aspect, about 10 to about 50 grams organic acid/day/gram of cells, in another aspect, about 14 to about 30 grams organic acid/day/gram of cells, in another aspect, about 2 to about 20 grams organic acid/day/gram of cells and in another aspect, about 15 to about 25 grams organic acid/day/gram of cells. In this aspect, the organic acid is acetic acid or butyric acid, or a mixture of both.

The organic acid 227 produced from the acetogenic CO₂ fermentation 220 may be sent to the CO fermentation 230 to increase alcohol productivity in the CO fermentation 230. In this aspect, the CO fermentation provides a specific alcohol productivity of about 16 grams alcohol/day/grams of cells or more, in another aspect, a specific alcohol productivity rate of about 18 g/day/grams of cells or more, in another aspect, a specific alcohol productivity rate of about 20 g/day/grams of cells or more, in another aspect, a specific alcohol productivity rate of about 22 g/day/grams of cells or more, in another aspect, a specific alcohol productivity rate of about 24 g/day/grams of cells or more, in another aspect, a specific alcohol productivity rate of about 16 to about 30 g/day/grams of cells, in another aspect, about 18 to about 34 g/day/grams of cells, in another aspect, about 20 to about 40 g/day/grams of cells, in another aspect, about 22 to about 48 g/day/grams of cells, in another aspect, about 20 to about 50 g/day/grams of cells, in another aspect, about 25 to about 50 g/day/grams of cells, and in another aspect, about 25 to about 55 g/day/grams of cells. In this aspect, the alcohol is ethanol or butanol, or a mixture of both.

The acetogenic CO₂ fermentation process is capable of providing a CO₂ conversion rate of 75% or more, in one aspect, 80% or more, in one aspect, 85% or more, in one aspect 90% or more, in another aspect, 85% to 95%, and still in another aspect, 90% to 99%. When the acetogenic CO₂ fermentation vessel 220 is connected with the CO fermentation vessel 230 and the methanogen fermentation vessel 105, the CO₂ conversion rate in the acetogenic CO₂ fermentation vessel 220 may be purposely lowered to balance the organic acid consumption in the CO fermentation vessel 230 and the CO₂ consumption in the acetogenic CO₂ fermentation vessel 220 and the methanogen fermentation vessel 105. In this scenario, the CO₂ conversion rate of the acetogenic CO₂ fermentation is controlled to 25% to 74%, in one aspect, 45% to 74%, in one aspect, 55 to 75%, in one aspect, 60 to 74%, in one aspect, 62 to 74%, and in another aspect, 65 to 74%. Vent gas 225 produced from the acetogenic CO₂ fermentation vessel 220 with controlled CO₂ conversion rate may contain 15 to 45% CO₂. The vent gas 225 may then be subsequently provided to methanogen fermentation vessel 105 to produce methane.

Alternatively, to balance the organic acid consumption in the CO fermentation vessel 230, vent gas 481 from the CO fermentation vessel 230 may be sent to both acetogenic CO₂ fermentation vessel 220 and methanogen fermentation vessel 105. In this scenario, CO₂ conversion rate in the acetogenic CO₂ fermentation vessel 220 is maintained at 75 to 99%. Vent gas 225 produced from the acetogenic CO₂ fermentation vessel 220 with high CO₂ conversion rate has a CO₂ concentration of 2 to 15%. Optionally, the vent gas 225 may be provided to methanogen fermentation vessel 105 to produce methane.

Further, the fermentation process can be manipulated under conditions that facilitate the production of desired product. In one aspect, the desired product is one or more oxygenated hydrocarbon compounds. In another aspect, the desired product is the microbial biomass itself, and the process produces oxygenated hydrocarbon compounds as byproducts. Operation parameters, such as culture medium flow rate, gaseous substrate feed rate, water supply/recycle rate, temperature, media redox potential, pressure, pH, agitation rate (if using a stirred tank reactor), and cell concentration, are monitored and controlled throughout the fermentation process.

A fermentation liquid broth is generated inside fermentation vessel once the acetogenic CO₂ fermentation process starts. In addition to the culture medium, the fermentation liquid broth also includes acetogenic bacteria and one or more oxygenated hydrocarbon compounds. In general, the fermentation liquid broth has a pH of about 8 or less, and in another aspect, about 7.5 or less. In one aspect, the cell concentration of the fermentation liquid broth is about 1 to about 15 g/L, in another aspect 2 to about 30 g/L, in another aspect, about 2 to about 25 g/L, in another aspect, about 2 to about 20 g/L, in another aspect, about 2 to about 10 g/L, in another aspect, about 2 to about 8 g/L, in another aspect, about 3 to about 30 g/L, in another aspect, about 3 to about 9 g/L, and in another aspect, about 4 to about 8 g/L.

CO Fermentation: As described herein in connection with FIG. 2, certain acetogenic bacteria can ferment CO-containing gaseous substrate 50 into useful oxygenated hydrocarbon compounds, such as ethanol and butanol 255, and Clostridium fermentation liquid broth 270. Acetogenic bacteria in the CO fermentation vessel 230 may also convert organic acids into oxygenated hydrocarbonaceous compounds in the presence of CO. As shown in both FIG. 3, vent gas 481 from the CO fermentation vessel 230 may be returned to the acetogenic CO₂ fermentation vessel 220 and/or to the Methanogen fermentation vessel. In this aspect, the vent gas 481 from the CO fermentation vessel 230 may be processed to remove CO prior to entering the methanogen fermentation vessel 105.

### Integrated Fermentation Systems

As would be understood by a person of ordinary skill in the art, any one or all of the processes described in FIGS. 1-3 may be combined into an overall system.

### Microbial Biomass

The fermentation liquid broth (FIG. 1: 140, FIGS. 2, 140 and 270, and FIGS. 3, 140, 250 and 270) from any of the fermentation vessels may be further purged out of the fermentation vessel and then separated into a cell-free permeate and a cell-containing suspension by one or more cell separators. Cell membranes of the cell-containing suspension are ruptured to generate a homogenate. The homogenate is fractionated into a protein-containing supernatant and a protein-containing cell debris portion using a fractionator.

Suitable cell separators include, but not limited to, filtration devices, hollow fiber filtration devices, spiral wound filtration devices, ultrafiltration devices, ceramic filter devices, cross-flow filtration devices, size exclusion column filtration devices, spiral wound membranes, centrifugation devices, and combination thereof. Processes for production of single cell proteins from biomass are described in U.S. Ser. No. 16/416,133, filed May 17, 2019,

The cell-containing suspension contains microbial cells at a cell concentration higher than the fermentation liquid broth. In one aspect, the cell concentration of the cell-containing suspension is about 20 g/L or more, in another aspect, about 30 g/L or more, in another aspect, about 40 g/L or more, in another aspect, about 50 g/L or more, in another aspect, about 60 g/L or more, in another aspect, about 20 to about 300 g/L, in another aspect, about 30 to about 250 g/L, in another aspect, about 40 to about 200 g/L, in another aspect, about 50 to about 150 g/L, in still another aspect, about 100 to about 150 g/L.

Cells of the cell-containing suspension may be ruptured using one or more rupturing devices selected from the group consisting of a microfluidics device, a sonication device, an ultrasonic device, a mechanical disruption device, a French press, a freezer, a heater, a heat exchanger, a distillation column, a pasteurization device, an UV sterilization device, a gamma ray sterilization device, a reactor, a homogenizer, and combinations thereof. In one aspect, a pH of the cell-containing suspension is adjusted to a pH of about 6 to about 12 before rupturing cell membranes of the cell-containing suspension, in another aspect, a pH of 7 to 12, in another aspect, a pH of 8 to 12, in another aspect, a pH of 7.5 to 11, in another aspect, a pH of 8.5 to 11, and in still another aspect, a pH of 7 to 10.

In another aspect, cell-containing suspension is hydrolyzed by a hydrolase enzyme. In this aspect, the cell-containing suspension and the hydrolase enzyme are incubated at a temperature of about 50 to about 70° C. for about 3 to about 72 hours to form a hydrolyzed lysate, in one aspect, 3 to 48 hours, in one aspect, 4 to 24 hours, in one aspect, 6 to 24 hours, in another aspect, 6 to 12 hours, and in still another aspect, 4 to 12 hours. pH of the cell-containing suspension is adjusted to a pH of about 6 to about 12 before hydrolysis of the cell-containing suspension, in another aspect, a pH of 7 to 12, in another aspect, a pH of 8 to 12, in another aspect, a pH of 7.5 to 11, in another aspect, a pH of 8.5 to 11, and in still another aspect, a pH of 7 to 10. The hydrolase enzyme is selected from the group consisting of subtilases, alcalase, serine protease, serine endopeptidase and mixtures thereof. The hydrolyzed lysate is fractionated into the protein-containing supernatant and the protein-containing cell debris portion using centrifugation, ultrafiltration, and combination thereof. The protein-containing supernatant has a nucleic acid content of less than about 5%, in one aspect, less than 4%, in one aspect, less than 3%, and in another aspect, less than 2%.

The protein-containing supernatant and the protein-containing cell debris portion may be directly used as or further processed to a protein containing nutrient supplement. A dehydration unit may be used to dry the protein-containing supernatant and produce a soluble protein containing nutrient supplement, such as protein powder. Suitable dehydration unit includes spray drying unit, drum dryer unit, freeze drying unit, lyophilizing unit, and combinations thereof. Other components, such as moisture and ash can be further removed to purify the protein containing supplement. The protein containing supplement may be directly used as animal feed or be blended with other ingredients for making into one or more types of nutrient supplements. In one aspect, the protein containing supplement contains about 60 to about 99 weight percent protein, in another aspect, about 70 to about 95 weight percent protein, in another aspect, about 75 to about 95 weight percent protein, in another aspect, about 80 to 95 weight percent protein, and in another aspect, about 85 to 95 weight percent protein.

### EXAMPLES

The following examples further illustrate the disclosure and should not be construed to limit its scope.

### Example 1: Methanothermobacter thermautotrophicus Fermentation

A gas containing CO₂ and H₂ is continuously introduced into a stirred tank bioreactor containing Methanothermobacter thermautotrophicus, along with a conventional liquid medium containing trace metals and salts. Vitamins are provided using a dedicated feed line.

New Brunswick Bioflow 320 reactor containing Fermentation Medium is started with actively growing Methanothermobacter thermautotrophicus. The rate of agitation of the reactor is set to 1200 rpm at the start of the experiment. This agitation rate is maintained throughout the experiment. Feed gas flow to the reactor is increased based on the H₂ and CO₂ uptake of the culture. Temperature in the bioreactor is maintained around 60° C. throughout the experiment. Samples of gas feed into the bioreactor and off-gas from the bioreactor and fermentation broth in the bioreactor are taken at intervals, for example feed gas, off-gas and fermentation broth are sampled about daily, once two hours and once four hours respectively. Above samples are analyzed for consumption or production of various gas components, and the optical density (cell density) of the culture. The unaroused volume of the reactor, i.e. the volume of the fermentation broth, is maintained at ˜2000-2200 ml throughout the experiment. Also, the gas flow to the reactor is measured real time by the mass flow controller regulating gas to the reactor. The feed gas composition of this experiment is 76% H₂, 20% CO₂ and 4% N₂.

In this experiment, a cell recycle system (CRS) is attached to the reactor before the start of the experiment. During the experiment, the rate of flow of nutrients (growth medium) to the reactor is 2.0 to 5.0 ml/min. The rate of culture purge is 3.0 to 5.0 ml/min and permeate is drawn out through the CRS at a rate of 0 to 2.0 ml/min.

Results can be summarized as follows:
Specific CO₂ uptake: 0.8 to 1.1 mmol CO₂/min/gram dry cells
Specific H₂ uptake: 3.3 to 3.8 mmol H₂/min/gram dry cells
Average Cell Retention Time: 11.2 hours
Average Cell Density: 3 g/L
CO₂ conversion rate: 90% to 99%
The effluent gas composition of this experiment is 13.8% H₂, 7.6% CO₂, 62.5% CH₄ and 16.1% N₂.
Specific methane productivity is 0.77 mmol/min/gram of cells.

### Example 2: Methanothermobacter marburgensis Fermentation

A gas containing CO₂ and H₂ is continuously introduced into a stirred tank bioreactor containing Methanothermobacter marburgensis, along with a conventional liquid medium containing trace metals and salts. Vitamins are provided using a dedicated feed line.

New Brunswick Bioflow 310 reactor containing Fermentation Medium is started with actively growing Methanothermobacter marburgensis. The rate of agitation of the reactor is set to 1200 rpm at the start of the experiment. This agitation rate is maintained throughout the experiment. Feed gas flow to the reactor is increased based on the H₂ and CO₂ uptake of the culture. Temperature in the bioreactor is maintained around 60° C. throughout the experiment. Samples of gas feed into the bioreactor and off-gas from the bioreactor and fermentation broth in the bioreactor are taken at intervals, for example feed gas, off-gas and fermentation broth are sampled about daily, once two hours and once four hours respectively. Above samples are analyzed for consumption or production of various gas components, and the optical density (cell density) of the culture. The unaroused volume of the reactor is maintained at ˜1500-1600 ml throughout the experiment. Also, the gas flow to the reactor is measured real time by the mass flow controller regulating gas to the reactor. The feed gas composition of this experiment is 70% H₂, 25% CO₂ and 5% N₂.

During the experiment, the rate of flow of nutrients (growth medium) to the reactor is 1.0-2.0 ml/min and through the culture purge pump, 1.0-2.0 ml/min culture is drawn out from the reactor.

Results can be summarized as follows:
Specific CO₂ uptake: 0.6 to 0.9 mmol CO₂/min/gram dry cells
Specific H₂ uptake: 2.8 to 3.2 mmol H₂/min/gram dry cells
Average Cell Retention Time: 18.2 hours
Average Cell Density: 3.4 g/L
CO₂ conversion rate: 65% to 85%
The effluent gas composition of this experiment is 7.4% H₂, 26.8% CO₂, 49.1% CH₄ and 16.7% N₂.
Specific methane productivity is 0.67 mmol/min/gram of cells

### Example 3: Effluent Gas Reforming

A portion of the effluent gas produced in Example 1 is sent to a methane reformer, which reforms methane into CO and H₂.

CH₄+H₂O (+heat)→CO+3H₂

Gas composition after reforming is 21.7% CO, 69.8% H₂, 2.7% CO₂, 0.2% CH₄, and 5.6% N₂. The gas after reforming is then separated by a H₂ removal unit into a CO rich syngas with 68.6% CO. 4.4% H₂, 8.4% CO₂, 0.7% CH₄, and 17.9% N₂ and a H₂ rich stream with 99% H₂ concentration.

### Example 4: Effluent Gas Pyrolysis

A portion of the effluent gas produced in Example 1 is sent to a gas separator to produce a methane concentrated feed gas with 2.6% H₂, 0.7% CO₂, 93.7% CH₄ and 3% N₂, and a methane diluted syngas 52% H₂, 32.6% CO₂, 6.7% CH₄, and 8.7% N₂. The methane concentrated feed gas is then passed to a microwave pyrolysis cracker to produce H₂ and elemental carbon.

CH₄ (+heat)→C+2H₂

### Example 5: Clostridium ljungdahlii and Methanothermobacter thermautotrophicus Fermentation

A synthesis gas containing CO, CO₂ and H₂ is continuously introduced into a stirred tank bioreactor containing Clostridium ljungdahlii, along with a liquid medium containing trace metals and salts as described herein. Vitamins are provided using dedicated feed lines.

A New Brunswick Bioflow reactor containing the fermentation medium is started with actively growing Clostridium ljungdahlii. The rate of agitation of the reactor is set to 800 rpm at the start of the experiment and this agitation rate is maintained throughout the experiment. Feed gas flow to the reactor is increased based on the H₂ and CO uptake of the culture. Temperature in the bioreactor is maintained at about 38° C. throughout the experiment. Samples of gas feed into the bioreactor and off-gas from the bioreactor and fermentation broth in the bioreactor are taken at intervals, for example feed gas, off-gas and fermentation broth were sampled about daily, once two hours and once four hours respectively. Above samples are analyzed for consumption or production of various gas components, broth acetic acid concentration, broth ethanol concentration and the optical density (cell density) of the culture. The unaroused volume of the reactor is maintained between 3000 to 3250 ml throughout the experiment. Further, the gas flow to the reactor is maintained at required gas flow rates with an average of 67.0 mmol/min by using a mass flow controller. The feed syngas composition is 23% H₂, 35% CO, 29% CO₂ and 13% N₂.

The following results are achieved during the fermentation:
Specific CO uptake: 0.9 to 1.3 mmol CO/min/gram of cells
Specific H₂ uptake: 0.2 to 0.6 mmol H₂/min/gram of cells
Average Cell Density: 5.85 g/L
Average Cell Retention Time: 8 hours
CO conversion rate: 90%
H₂ conversion rate: 30%
Specific ethanol productivity: 13.31 gram/day/gram of cells
Vent gas composition: 4% CO, 20% H₂, 59% CO₂, and 16% N₂
Vent gas flow rate: 52.95 mmol/min

The vent gas is then blended with an H₂ stream and fed to a Methanothermobacter thermautotrophicus fermentation reactor. The Methanothermobacter thermautotrophicus fermentation is performed as illustrated in Example 1. A two-reactor process is configured as shown in FIG. 2. Blended feed gas composition: 1.4% CO, 74.9% H₂, 18.5% CO₂, and 5.2% N₂.

The following results are achieved during the fermentation:
Specific CO₂ uptake: 0.8 to 1.3 mmol CO₂/min/gram of cells
Specific H₂ uptake: 3.2 to 4.5 mmol H₂/min/gram of cells
Average Cell Retention Time: 10 hours
Average Cell Density: 3 g/L
CO₂ conversion: 90% to 99%
Specific methane productivity: 0.83 mmol/min/gram of cells
Effluent gas composition: 9.8% H₂, 1.7% CO₂, 67.6% CH₄, and 20.9% N₂.
Effluent gas flow rate: 7.4 mmol/min

Average gas composition and gas flow rate are as follows:

| | | Flow Rate |
|---|---|---|
| | Composition | (mmol/min) |
| Clostridium ljungdahlii feed gas | 23% H₂, 35% CO,29% CO₂, 13% N₂. | 67 |
| Clostridium ljungdahlii vent gas | 4% CO, 20% H₂, 59% CO₂, 16% N₂ | 52.95 |
| Methanothermobacter thermautotrophicus | 1.4% CO, 74.9% H₂, 18.5% CO₂, 5.2% N₂ | 29.8 |
| feed gas | | |
| Methanothermobacter thermautotrophicus | 9.8% H₂, 1.7% CO₂, 67.6% CH₄, 20.9% N₂ | 7.4 |
| effluent gas | | |

A portion of effluent gas may then be sent to a methane reformer as illustrated in Example 3 to produce a CO rich syngas and a H₂ rich stream. The CO rich syngas can then be blended with the synthesis gas to provide a blended syngas feed gas to the Clostridium ljungdahlii fermentation reactor and the H₂ rich stream can be used to be blended with the vent gas from the Clostridium ljungdahlii fermentation.

A comparison of percentages in gas compositions illustrates the following:
The percentage of CO from high of 35% to a low of 0) %. In this aspect, the percent reduction of CO may range from 90 to 100%, in another aspect, 95 to 100%, and in another aspect, 99 to 100%.
The percentage of CO₂ from a high of 29% to a low of 1.7%. In this aspect, the percent reduction of CO₂ may range from 90 to 95%, in another aspect, 92 to 95%, and in another aspect, 94 to 95%.

### Example 6: Clostridium ljungdahlii, Acetobacterium woodii and Methanothermobacter thermautotrophicus Fermentation

A stirred tank New Brunswick Bioflow bioreactor containing actively growing Acetobacterium woodii along with a growth medium is added to the system as illustrated in Example 5 to receive the vent gas from the Clostridium ljungdahlii fermentation bioreactor. The rate of agitation of the Acetobacterium woodie bioreactor is set to 600 rpm. This agitation rate remains constant throughout the experiment. Feed gas flow to the reactor is maintained at 36.6 mL/min to 44.4 mL/min. Temperature in the bioreactor is maintained at 33° C. throughout the experiment. Na+ levels are kept at 3500 to 4000 ppm. Samples of gas feed into the bioreactor and off-gas from the bioreactor and fermentation broth in the bioreactor are taken at intervals, for example feed gas, offs-gas and fermentation broth is sampled about daily, once two hours and once four hours respectively. Above samples are analyzed for consumption or production of various gas components, broth, acetic acid concentration, and the optical density (cell density) of the culture. The unaroused volume of the reactor was maintained between 1900 to 2275 ml throughout the experiment. Also, the gas flow to the reactor is maintained at required gas flow rates using a mass flow controller.

A cell recycle system (CRS) is attached to the reactor before the start of the experiment During the experiment, the rate of flow of nutrients (growth medium) to the reactor is maintained at 2.8 ml/min. Medium feed rate was maintained throughout the experiment. The average rate of base (NaOH) requirement to maintain pH at 6.5 is 0.075 ml/min, and through the CRS, 2.9 ml/min permeate is drawn out from the reactor.

Vent gas from the Acetobacterium woodii bioreactor, blended with a supplemented H₂ stream, is sent to the Methanothermobacter thermautotrophicus fermentation bioreactor. Acetic acid produced from the Acetobacterium woodii bioreactor is sent to the Clostridium ljungdahlii fermentation bioreactor. CO₂ conversion rate of the Acetobacterium woodii fermentation achieved 90 to 97% at the beginning. After the water loop between the Acetobacterium woodii bioreactor and the Clostridium ljundahlii bioreactor is closed, CO₂ conversion rate of the Acetobacterium woodii fermentation is purposely lowered to 60 to 70%. The Methanothermobacter thermautotrophicus fermentation and the Clostridium ljungdahlii fermentation are performed as illustrated in Example 5. A three-reactor process is configured as shown in FIG. 3. Feed gas composition to the Clostridium ljungdahlii fermentation: 36% CO, 63% H₂, 0% CO₂, and 1% N₂

The following results are achieved during the Clostridium ljungdahlii fermentation:
Specific CO uptake: 1.3 to 1.7 mmol CO/min/gram of cells
Specific H₂ uptake: 0.2 to 0.6 mmol H₂/min/gram of cells
Average Cell Density: 6.43 g/L
Average Cell Retention Time: 7.2 hours
CO conversion rate: 90%
H₂ conversion rate: 14%
Specific ethanol productivity (before water loop closure): 13.43 gram/day/gram of cells
Specific ethanol productivity (after water loop closure) 36.87 gram/day/gram of cells
Vent gas composition: 4% CO, 67% H₂, 27% CO₂, and 1% N₂
Vent gas flow rate: 71.77 mmol/min

The following results are achieved during the Acetobacterium woodii fermentation:
Specific CO₂ uptake: 0.4 to 0.9 mmol CO/min/gram of cells
Specific H₂ uptake: 1.0 to 1.5 mmol H₂/min/gram of cells
Average Cell Density: 6.3 g/L
Average Cell Retention Time: 20 hours
CO conversion rate: 100%
CO₂ conversion rate: 60%
H₂ conversion rate: 48%

Specific acetic acid productivity: 30.55 gram/day/gram of cells
Vent gas composition: 0% CO, 74.6% H₂, 22.8% CO₂, and 2.6% N₂
Vent gas flow rate: 33.84 mmol/min

The following results are achieved during the Methanothermobacter thermautotrophicus fermentation:
Specific CO₂ uptake: 0.6 to 1.1 mmol CO/min/gram of cells
Specific H₂ uptake: 3.3 to 4.0 mmol H₂/min/gram of cells
Average Cell Density: 4.24 g/L
Average Cell Retention Time: 11 hours
CO₂ conversion rate: 99%
H₂ conversion rate: 99%
Specific methane productivity: 0.77 mmol/min/gram of cells
Effluent gas composition: 0% CO, 4% H₂, 1% CO₂, 84% CH₄ and 11% N₂
Effluent gas flow rate: 7.86 mmol/min

Average gas composition and gas flow rate are as follows:

| | | Flow Rate |
|---|---|---|
| | Composition | (mmol/min) |
| Clostridium ljungdahlii feed gas | 36% CO, 63% H₂, 0% CO₂, 1% N₂ | 89.3 |
| Clostridium ljungdahlii vent gas | 4% CO, 67% H₂, 27% CO₂, 1% N₂ | 71.77 |
| Acetobacterium woodii feed gas | 4% CO, 67% H₂, 27% CO₂, 1% N₂ | 71.77 |
| Acetobacterium woodii vent gas | 0% CO, 74.6% H₂, 22.8% CO₂, 2.6% N₂ | 33.84 |
| Methanothermobacter thermautotrophicus | 0% CO, 78% H₂, 19% CO₂, 2% N₂ | 39.9 |
| feed gas | | |
| Methanothermobacter thermautotrophicus | 0% CO, 4% H₂, 1% CO₂, 84% CH₄, 11% N₂ | 7.86 |
| effluent gas | | |

A comparison of percentages in gas compositions illustrates the following:
The percentage of CO from high of 36% to a low of 0%. In this aspect, the percent reduction of CO may range from 90 to 100%, in another aspect, 95 to 100%, and in another aspect, 99 to 100%.
The percentage of CO₂ from a high of 27% to a low of 1%. In this aspect, the percent reduction of CO₂ may range from 90 to 97%, in another aspect, 95 to 97%, and in another aspect, 96 to 97%.

While the disclosure herein disclosed has been described by means of specific embodiments, examples, and applications thereof, other and further variations could be devised without departing from the basic scope of the disclosure set forth in the claims that follow.

What is claimed is:

1. A process for converting CO₂, the process comprising:
fermenting a gaseous substrate that includes CO₂ and H₂ with methanogenic archaea in a methanogen fermentation vessel, wherein a CO₂ to H₂ ratio of about 1:3 to about 1:4 is maintained in the gaseous substrate; and
recovering methane from the methanogen fermentation vessel.

2. The process of claim 1 wherein the methanogenic archaea is selected from the group consisting of Methanobacterium alcaliphilum, Methanobacterium bryantii, Methanobacterium congolense, Methanobacterium defluvii, Methanobacterium espanolae, Methanobacterium formicicum, Methanobacterium ivanovii, Methanobacterium palustre, Methanobacterium thermaggregans, Methanobacterium uliginosum, Methanobrevibacter acididurans, Methanobrevibacter arboriphilicus, Methanobrevibacter gottschalkii, Methanobrevibacter olleyae, Methanobrevibacter ruminantium, Methanobrevibacter smithii, Methanobrevibacter woesei, Methanobrevibacter wolinii, Methanothermobacter marburgensis, Methanothermobacter thermautotrophicum, Methanothermobacter thermoflexus, Methanothermobacter thermophilus, Methanothermobacter wolfeii, Methanothermus sociabilis, Methanocorpusculum bavaricum, Methanocorpusculum parvum, Methanoculleus chikuoensis, Methanoculleus submarinus, Methanogenium frigidum, Methanogenium liminatans, Methanogenium marinum, Methanosarcina acetivorans, Methanosarcina barkeri, Methanosarcina mazei, Methanosarcina thermophila, Methanomicrobium mobile, Methanocaldococcus jannaschii, Methanococcus aeolicus, Methanococcus maripaludis, Methanococcus vannielii, Methanococcus voltaei, Methanothermococcus thermolithotrophicus, Methanopyrus kandleri, Methanothermobacter thermoautotroiphicus, Methanocaldococcus fervens, Methanocaldococcus indicus, Methanocaldococcus infernus, and Methanocaldococcus vulcanius.

3. The process of claim 1 wherein the methanogenic archaea is Methanothermobacter thermoautotroiphicus.

4. The process of claim 1 wherein the process provides a specific CO₂ uptake of about 0.5 to about 3 mmol CO₂/minute/gram of cells.

5. The process of claim 1 wherein the process provides a specific H₂ uptake of about 1.5 to about 12 mmol H₂/minute/gram of cells.

6. The process of claim 1 wherein the process has a cell retention time of about 5 to about 50 hours.

7. The process of claim 1 wherein the process provides a methane productivity of about 0.5 to about 2.5 mmol methane/minute/gram of cells.

8. The process of claim 1 wherein the process provides a CO₂ conversion rate of 65% or more.

9. The process of claim 1 further comprising:
obtaining from the methanogen fermentation vessel a fermentation liquid broth containing methanogenic archaea;
separating the fermentation liquid broth into a cell-free permeate and a cell-containing suspension;
rupturing cell membranes of the cell-containing suspension to generate a homogenate;
fractionating the homogenate into a protein-containing supernatant and a protein-containing cell debris portion using a fractionator; and
obtaining a protein containing nutrient supplement.

10. The process of claim 9 wherein the cell-containing suspension has a dry cell weight concentration of about 50 g/liter to about 200 g/liter.

11. The process of claim 9 wherein rupturing cell membranes of the cell-containing suspension is conducted using one or more rupturing devices selected from the group consisting of a microfluidics device, a sonication device, an ultrasonic device, a mechanical disruption device, a French press, a freezer, a heater, a heat exchanger, a distillation column, a pasteurization device, an UV sterilization device, a gamma ray sterilization device, a reactor, a homogenizer, and combinations thereof.

12. The process of claim 9 wherein pH of the cell-containing suspension is adjusted to a pH of about 6 to about 12 before rupturing cell membranes of the cell-containing suspension.

13. The process of claim 9 wherein the homogenate is a hydrolyzed lysate formed by contacting the cell-containing suspension with a hydrolase enzyme.

14. The process of claim 13 wherein the cell-containing suspension and the hydrolase enzyme are incubated at a temperature of about 50 to about 70° C. for about 3 to about 72 hours to form the hydrolyzed lysate.

15. The process of claim 13 wherein the hydrolase enzyme is selected from the group consisting of subtilases, alcalase, serine protease, serine endopeptidase and mixtures thereof.

16. The process of claim 9 wherein the homogenate is fractionated into the protein-containing supernatant and the protein-containing cell debris portion using centrifugation, ultrafiltration, and combination thereof.

17. The process of claim 9 wherein the protein-containing supernatant has a nucleic acid content of less than about 5%.

18. The process of claim 9 wherein the protein-containing supernatant is dehydrated to provide a soluble protein containing nutrient supplement with about 60 to about 99 dry weight percent protein.

19. A process for converting CO₂, the process comprising:
fermenting a gaseous substrate that includes CO₂ and H₂ with methanogenic archaea in a methanogen fermentation vessel;
recovering methane from the methanogen fermentation vessel;
cracking at least a portion of the methane to generate H₂; and
returning at least a portion of the H₂ to the methanogen fermentation vessel.

20. The process of claim 19 wherein a CO₂ to H₂ ratio of about 1:3 to about 1:4 is maintained in the gaseous substrate.

21. The process of claim 19 wherein the methanogenic archaea is Methanothermobacter thermoautotroiphicus.

22. The process of claim 19 wherein the process provides a specific CO₂ uptake of about 0.5 to about 3 mmol CO₂/minute/gram of cells.

23. The process of claim 19 wherein the process provides a specific H₂ uptake of about 1.5 to about 12 mmol H₂/minute/gram of cells.

24. The process of claim 19 wherein the process has a cell retention time of about 5 to about 50 hours.

25. The process of claim 19 wherein the process provides a methane productivity of about 0.5 to about 2.5 mmol methane/minute/gram of cells.

26. The process of claim 19 wherein the process provides a CO₂ conversion rate of 65% or more.

27. The process of claim 19 wherein the cracking is conducted in a methane cracker selected from the group consisting of a microwave pyrolysis cracker, a molten metal pyrolysis cracker, a plasma arc pyrolysis cracker and combinations thereof.

28. The process of claim 19 wherein the cracking is conducted in a methane cracker selected from the group consisting of a steam reformer, a dry reformer, a partial oxidation reformer and combinations thereof.

29. The process of claim 19 further comprising:
obtaining from the methanogen fermentation vessel a fermentation liquid broth containing methanogenic archaea;
separating the fermentation liquid broth into a cell-free permeate and a cell-containing suspension;
rupturing cell membranes of the cell-containing suspension to generate a homogenate;
fractionating the homogenate into a protein-containing portion and a protein-containing cell debris portion using a fractionator; and
obtaining a protein containing nutrient supplement.

30. A process for converting CO and CO₂, the process comprising:
fermenting a gaseous substrate that includes CO and H₂ with CO converting acetogenic bacteria in a CO fermentation vessel to produce an alcohol and a CO₂ containing vent gas;
providing the CO₂ containing vent gas from the CO fermentation vessel to a methanogen fermentation vessel; and
fermenting the CO₂ containing vent gas with methanogenic archaea in the methanogen fermentation vessel to produce methane.

31. The process of claim 30 wherein the gaseous substrate contains at least 20 mole % CO.

32. The process of claim 30 wherein the methanogenic archaea is selected from the group consisting of Methanobacterium alcaliphilum, Methanobacterium bryantii, Methanobacterium congolense, Methanobacterium defluvii, Methanobacterium espanolae, Methanobacterium formicicum, Methanobacterium ivanovii, Methanobacterium palustre, Methanobacterium thermaggregans, Methanobacterium uliginosum, Methanobrevibacter acididurans, Methanobrevibacter arboriphilicus, Methanobrevibacter gottschalkii, Methanobrevibacter olleyae, Methanobrevibacter ruminantium, Methanobrevibacter smithii, Methanobrevibacter woesei, Methanobrevibacter wolinii, Methanothermobacter marburgensis, Methanothermobacter thermautotrophicum, Methanothermobacter thermoflexus, Methanothermobacter thermophilus, Methanothermobacter wolfeii, Methanothermus sociabilis, Methanocorpusculum bavaricum, Methanocorpusculum parvum, Methanoculleus chikuoensis, Methanoculleus submarinus, Methanogenium frigidum, Methanogenium liminatans, Methanogenium marinum, Methanosarcina acetivorans, Methanosarcina barkeri, Methanosarcina mazei, Methanosarcina thermophila, Methanomicrobium mobile, Methanocaldococcus jannaschii, Methanococcus aeolicus, Methanococcus maripaludis, Methanococcus vannielii, Methanococcus voltaei, Methanothermococcus thermolithotrophicus, Methanopyrus kandleri, Methanothermobacter thermoautotroiphicus, Methanocaldococcus fervens, Methanocaldococcus indicus, Methanocaldococcus infernus, and Methanocaldococcus vulcanius.

33. The process of claim 30 wherein the methanogenic archaea is Methanothermobacter thermoautotroiphicus.

34. The process of claim 30 wherein the methanogen fermentation vessel provides a specific CO₂ uptake of about 1 to about 3 mmol CO₂/minute/gram of cells.

35. The process of claim 30 wherein the methanogen fermentation vessel provides a specific H₂ uptake of about 3 to about 12 mmol H₂/minute/gram of cells.

36. The process of claim 30 wherein the methanogen fermentation vessel has a cell retention time of about 5 to about 50 hours.

37. The process of claim 30 wherein the methanogen fermentation vessel provides a methane productivity of about 1.2 to about 2.5 mmol methane/minute/gram of cells.

38. The process of claim 30 wherein the methanogen fermentation vessel provides a CO₂ conversion rate of 65% or more.

39. The process of claim 30 wherein the CO converting acetogenic bacteria is selected from the group consisting of Clostridium aceticum, Clostridium acetobutylicum, Clostridium acetobutylicum P262, Clostridium autoethanogenum (DSM 19630 of DSMZ Germany), Clostridium autoethanogenum (DSM 10061 of DSMZ Germany), Clostridium autoethanogenum (DSM 23693 of DSMZ Germany), Clostridium autoethanogenum (DSM 24138 of DSMZ Germany), Clostridium carboxidivorans, Clostridium coskatii (ATCC PTA-10522), Clostridium drakei, Clostridium ljungdahlit PETC (ATCC 49587), Clostridium ljungdahlii ERI2 (ATCC 55380), Clostridium ljungdahlii C-01 (ATCC 55988), Clostridium ljungdahlii O-52 (ATCC 55889), Clostridium magnum, Clostridium pasteurianum (DSM 525 of DSMZ Germany), Clostridium ragsdalei P11 (ATCC BAA-622), Clostridium scatologenes, Clostridium thermoaceticum, Clostridium ultunense, Clostridium Stick-landii, and mixtures thereof.

40. The process of claim 30 wherein the alcohol is ethanol.

41. The process of claim 30 wherein the CO fermentation vessel provides a CO conversion rate of 80% or more.

42. The process of claim 30 further comprising:
providing the methane from the methanogen fermentation vessel to a methane cracker to produce a H₂ rich stream and a CO rich syngas,
supplying the CO rich syngas to the CO fermentation vessel, and
supplying H₂ rich stream to the methanogen fermentation vessel.

43. The process of claim 30 further comprising:
providing the methane from the methanogen fermentation vessel to a methane cracker to produce a H₂ and CO containing gaseous substrate, and
supplying the H₂ and CO containing gaseous substrate to the CO fermentation vessel.

44. The process of claim 30 further comprising:
obtaining from the CO fermentation vessel a fermentation liquid broth containing CO converting acetogenic bacteria and from the methanogen fermentation vessel a fermentation liquid broth containing methanogenic archaea;
separating the fermentation liquid broth containing CO converting acetogenic bacteria and the fermentation liquid broth containing methanogenic archaea into a cell-free permeate and a cell-containing suspension;
rupturing cell membranes of the cell-containing suspension to generate a homogenate;
fractionating the homogenate into a protein-containing supernatant and a protein-containing cell debris portion using a fractionator; and
obtaining a protein containing nutrient supplement.

45. The process of claim 44 wherein the cell-containing suspension has a dry cell weight concentration of about 20 g/liter to about 200 g/liter.

46. The process of claim 44 wherein rupturing cell membranes of the cell-containing suspension is conducted using one or more rupturing devices selected from the group consisting of a microfluidics device, a sonication device, an ultrasonic device, a mechanical disruption device, a French press, a freezer, a heater, a heat exchanger, a distillation column, a pasteurization device, an UV sterilization device, a gamma ray sterilization device, a reactor, a homogenizer, and combinations thereof.

47. The process of claim 44 wherein pH of the cell-containing suspension is adjusted to a pH of about 6 to about 12 before rupturing cell membranes of the cell-containing suspension.

48. The process of claim 44 wherein the homogenate is a hydrolyzed lysate formed by contacting the cell-containing suspension with a hydrolase enzyme.

49. The process of claim 48 wherein the cell-containing suspension and the hydrolase enzyme are incubated at a temperature of about 50 to about 70° C. for about 3 to about 72 hours to form a hydrolyzed lysate.

50. The process of claim 48 wherein the hydrolase enzyme is selected from the group consisting of subtilases, alcalase, serine protease, serine endopeptidase and mixtures thereof.

51. The process of claim 44 wherein the homogenate is fractionated into the protein-containing supernatant and the protein-containing cell debris portion using centrifugation, ultrafiltration, and combination thereof.

52. The process of claim 44 wherein the protein-containing supernatant has a nucleic acid content of less than about 5%.

53. The process of claim 44 wherein the protein-containing supernatant is dehydrated to provide a soluble protein containing nutrient supplement with about 60 to about 99 dry weight percent protein.

54. A process for converting CO and CO₂, the process comprising:
fermenting a gaseous substrate that includes CO and H₂ with CO converting acetogenic bacteria in a CO fermentation vessel to produce an alcohol and a first CO₂ containing vent gas;
providing the first CO₂ containing vent gas from the CO fermentation vessel to an acetogenic CO₂ fermentation vessel;
fermenting the first CO₂ containing vent gas with CO₂ converting acetogenic bacteria in the acetogenic CO₂ fermentation vessel to produce an organic acid and a second CO₂ containing vent gas;
providing the second CO₂ containing vent gas to a methanogen fermentation vessel and the organic acid to the CO fermentation vessel; and
fermenting the second CO₂ containing vent gas with methanogenic archaea in the methanogen fermentation vessel to produce methane.

55. The process of claim 54 wherein the methanogenic archaea is selected from the group consisting of Methanobacterium alcaliphilum, Methanobacterium bryantii, Methanobacterium congolense, Methanobacterium defluvii, Methanobacterium espanolae, Methanobacterium formicicum, Methanobacterium ivanovii, Methanobacterium palustre, Methanobacterium thermaggregans, Methanobacterium uliginosum, Methanobrevibacter acididurans, Methanobrevibacter arboriphilicus, Methanobrevibacter gottschalkii, Methanobrevibacter olleyae, Methanobrevibacter ruminantium, Methanobrevibacter smithii, Methanobrevibacter woesei, Methanobrevibacter wolinii, Methanothermobacter marburgensis, Methanothermobacter thermautotrophicum, Methanothermobacter thermoflexus, Methanothermobacter thermophilus, Methanothermobacter wolfeii, Methanothermus sociabilis, Methanocorpusculum bavaricum, Methanocorpusculum parvum, Methanoculleus chikuoensis, Methanoculleus submarinus, Methanogenium frigidum, Methanogenium liminatans, Methanogenium marinum, Methanosarcina acetivorans, Methanosarcina barkeri, Methanosarcina mazei, Methanosarcina thermophila, Methanomicrobium mobile, Methanocaldococcus jannaschii, Methanococcus aeolicus, Methanococcus maripaludis, Methanococcus vannielii, Methanococcus voltaei, Methanothermococcus thermolithotrophicus, Methanopyrus kandleri, Methanothermobacter thermoautotroiphicus, Methanocaldococcus fervens, Methanocaldococcus indicus, Methanocaldococcus infernus, and Methanocaldococcus vulcanius.

56. The process of claim 54 wherein the methanogenic archaea is Methanothermobacter thermoautotroiphicus.

57. The process of claim 54 wherein the methanogen fermentation vessel provides a specific CO₂ uptake of about 1 to about 3 mmol CO₂/minute/gram of cells.

58. The process of claim 54 wherein the methanogen fermentation vessel provides a specific H₂ uptake of about 3 to about 12 mmol H₂/minute/gram of cells.

59. The process of claim 54 wherein the methanogen fermentation vessel has a cell retention time of about 5 to about 50 hours.

60. The process of claim 54 wherein the methanogen fermentation vessel provides a methane productivity of about 1.2 to about 2.5 mmol methane/minute/gram of cells.

61. The process of claim 54 wherein the methanogen fermentation vessel provides a CO₂ conversion rate of 65% or more.

62. The process of claim 54 wherein the methanogen fermentation vessel receives a supplemented H₂ stream.

63. The process of claim 54 wherein the acetogenic CO₂ fermentation vessel receives a supplemented H₂ stream.

64. The process of claim 54 wherein the CO₂ converting acetogenic bacteria is selected from the group consisting of Acetogenium kivui, Acetoanaerobium noterae, Acetobacterium woodii, Alkalibaculum bacchi, Acetobacterium bakii, and mixtures thereof.

65. The process of claim 54 wherein the organic acid is one or more C1 to C10 organic acids.

66. The process of claim 54 wherein the organic acid is acetic acid.

67. The process of claim 54 wherein CO₂ conversion rate in the acetogenic CO₂ fermentation vessel is controlled to 55% to 75%.

68. The process of claim 54 wherein the CO converting acetogenic bacteria is selected from the group consisting of Clostridium aceticum, Clostridium acetobutylicum, Clostridium acetobutylicum P262, Clostridium autoethanogenum (DSM 19630 of DSMZ Germany), Clostridium autoethanogenum (DSM 10061 of DSMZ Germany), Clostridium autoethanogenum (DSM 23693 of DSMZ Germany), Clostridium autoethanogemum (DSM 24138 of DSMZ Germany), Clostridium carboxidivorans, Clostridium coskatii (ATCC PTA-10522), Clostridium drakei, Clostridium ljungdahlii PETC (ATCC 49587), Clostridium ljungdahlii ERI2 (ATCC 55380), Clostridium ljungdahlii C-01 (ATCC 55988), Clostridium ljungdahlii O-52 (ATCC 55889), Clostridium magnum, Clostridium pasteurianum (DSM 525 of DSMZ Germany), Clostridium ragsdalei P11 (ATCC BAA-622), Clostridium scatologenes, Clostridium thermoaceticum, Clostridium ultunense, Clostridium Stick-landii, and mixtures thereof.

69. The process of claim 54 wherein the alcohol is ethanol.

70. The process of claim 54 wherein the CO fermentation vessel provides a CO conversion rate of 80% or more.

71. The process of claim 54 wherein the CO fermentation vessel provides a specific alcohol productivity of 10 grams alcohol/day/per gram of cells or more.

72. The process of claim 54 further comprising:
obtaining from the CO fermentation vessel a fermentation liquid broth containing CO converting acetogenic bacteria, from the acetogenic CO₂ fermentation vessel a fermentation liquid broth containing CO₂ converting acetogenic bacteria, and from the methanogen fermentation vessel a fermentation liquid broth containing methanogenic archaea;
separating the fermentation liquid broth containing CO converting acetogenic bacteria, the fermentation liquid broth containing CO₂ converting acetogenic bacteria and the fermentation liquid broth containing methanogenic archaea into a cell-free permeate and a cell-containing suspension;
rupturing cell membranes of the cell-containing suspension to generate a homogenate;
fractionating the homogenate into a protein-containing supernatant and a protein-containing cell debris portion using a fractionator; and
obtaining a protein containing nutrient supplement.

73. The process of claim 72 wherein the cell-containing suspension has a dry cell weight concentration of about 20 g/liter to about 200 g/liter.

74. The process of claim 72 wherein rupturing cell membranes of the cell-containing suspension is conducted using one or more rupturing devices selected from the group consisting of a microfluidics device, a sonication device, an ultrasonic device, a mechanical disruption device, a French press, a freezer, a heater, a heat exchanger, a distillation column, a pasteurization device, an UV sterilization device, a gamma ray sterilization device, a reactor, a homogenizer, and combinations thereof.

75. The process of claim 72 wherein pH of the cell-containing suspension is adjusted to a pH of about 6 to about 12 before rupturing cell membranes of the cell-containing suspension.

76. The process of claim 72 wherein the homogenate is a hydrolyzed lysate formed by contacting the cell-containing suspension with a hydrolase enzyme.

77. The process of claim 76 wherein the cell-containing suspension and the hydrolase enzyme are incubated at a temperature of about 50 to about 70° C. for about 3 to about 72 hours to form a hydrolyzed lysate.

78. The process of claim 76 wherein the hydrolase enzyme is selected from the group consisting of subtilases, alcalase, serine protease, serine endopeptidase and mixtures thereof.

79. The process of claim 72 wherein the homogenate is fractionated into the protein-containing supernatant and the protein-containing cell debris portion using centrifugation, ultrafiltration, and combination thereof.

80. The process of claim 72 wherein the protein-containing supernatant has a nucleic acid content of less than about 5%.

81. The process of claim 72 wherein the protein-containing supernatant is dehydrated to provide a soluble protein containing nutrient supplement with about 60 to about 99 dry weight percent protein.

82. A process for converting CO and CO₂, the process comprising:
fermenting a gaseous substrate that includes CO and H₂ with CO converting acetogenic bacteria in a CO fermentation vessel to produce an alcohol and a first CO₂ containing vent gas;
providing at least a portion of the first CO₂ containing vent gas from the CO fermentation vessel to an acetogenic CO₂ fermentation vessel;
fermenting the at least a portion of the first CO₂ containing vent gas with CO₂ converting acetogenic bacteria in the acetogenic CO₂ fermentation vessel to produce an organic acid;
providing the organic acid to the CO fermentation vessel;
providing at least another portion of the first CO₂ containing vent gas from the CO fermentation vessel to a methanogen fermentation vessel; and
fermenting the at least another portion of the first CO₂ containing vent gas with methanogenic archaea in the methanogen fermentation vessel to produce methane.

83. The process of claim 82 wherein the acetogenic CO₂ fermentation vessel further produces a second CO₂ containing vent gas and the second CO₂ containing vent gas is provided to the methanogen fermentation vessel.

84. The process of claim 82 wherein the methanogen fermentation vessel provides a methane productivity of about 1.2 to about 2.5 mmol methane/minute/gram of cells.

85. The process of claim 82 wherein the methanogen fermentation vessel provides a CO₂ conversion rate of 65% or more.

86. The process of claim 82 wherein CO₂ conversion rate in the acetogenic CO₂ fermentation vessel is controlled to 55% to 75%.

87. The process of claim 82 wherein the CO fermentation vessel provides a CO conversion rate of 80% or more.

88. The process of claim 82 wherein the CO fermentation vessel provides a specific alcohol productivity of 10 grams alcohol/day/per gram of cells or more.

89. The process of claim 82 further comprising:
obtaining from the CO fermentation vessel a fermentation liquid broth containing CO converting acetogenic bacteria, from the acetogenic CO₂ fermentation vessel a fermentation liquid broth containing CO₂ converting acetogenic bacteria, and from the methanogen fermentation vessel a fermentation liquid broth containing methanogenic archaea;
separating the fermentation liquid broth containing CO converting acetogenic bacteria, the fermentation liquid broth containing CO₂ converting acetogenic bacteria and the fermentation liquid broth containing methanogenic archaea into a cell-free permeate and a cell-containing suspension;
rupturing cell membranes of the cell-containing suspension to generate a homogenate;
fractionating the homogenate into a protein-containing supernatant and a protein-containing cell debris portion using a fractionator; and
obtaining a protein containing nutrient supplement.
